(19) United States
(12) Patent Application Publication
(10) Pub. No.: US 2025/0256076 A1
(43) Pub. Date: Aug. 14, 2025

(54) MULTI-MODAL PHYSIOLOGICAL SENSOR

(71) Applicant: NEUFLUENT LLC, Seattle, WA (US)

(72) Inventors: Samuel Robert Browd, Mercer Island, WA (US); Ryan J. Douglas, Seattle, WA (US)

(73) Assignee: NEUFLUENT LLC, Seattle, WA (US)

(21) Appl. No.: 19/052,113

(22) Filed: Feb. 12, 2025

Related U.S. Application Data
(60) Provisional application No. 63/552,894, filed on Feb. 13, 2024.
(60) Provisional application No. 63/552,563, filed on Feb. 12, 2024.

(51) Int. Cl. A61M 27/00

(52) U.S. Cl. CPC A61M 27/006; A61M 2202/0014; A61M 2202/04; A61M 2205/0216; A61M 2205/04; A61M 2205/18; A61M 2205/3303; A61M 2205/3327; A61M 2205/3334; A61M 2205/3344; A61M 2205/52; A61M 2210/0687; A61M 2230/10

(57) ABSTRACT

Apparatus and associated methods relate to a multimodal signal management system (MMSMS) that includes two or more electrodes, and a cerebrospinal fluid pressure (CSF) monitor enclosed within a housing implanted in the brain. In an illustrative example, the MMSMS, may, for example, include a conduit configured to provide fluid communication between the CSF-containing region in the brain and a remote location. Various embodiments may advantageously provide therapeutical CSF draining while monitoring electroencephalogram (EEG) and CSF pressure waveform information.

### CROSS-REFERENCE TO RELATED APPLICATIONS

This application is a nonprovisional application and claims the benefit of U.S. Provisional Application Ser. No. 63/552,563, titled “MULTI-MODAL PHYSIOLOGICAL SENSOR,” filed by Samuel Robert Browd on Feb. 12, 2024.

This application also claims the benefit of U.S. Provisional Application Ser. No. 63/552,894, titled “CONTINUOUS NEUROCARE,” filed by Samuel Robert Browd and Ryan J. Douglas on Feb. 13, 2024.

This application incorporates the entire contents of the foregoing application(s) herein by reference.

The subject matter of this application may have common inventorship with and/or may be related to the subject matter of the following:
U.S. Provisional application Ser. No. 63/364,253, titled “SHUNT TECHNOLOGY AND THE POTENTIAL FOR A SMART SHUNT,” filed by Samuel Robert Browd on May 5, 2022.
U.S. Provisional application Ser. No. 63/447,158, titled “SHUNT TECHNOLOGY AND THE POTENTIAL FOR A SMART SHUNT,” filed by Samuel Robert Browd and Ryan J. Douglas on Dec. 23, 2022.
U.S. Provisional application Ser. No. 63/447,162, titled “Cerebrospinal Fluid Polarization,” filed by Samuel Robert Browd and Ryan J. Douglas on Dec. 23, 2022.
U.S. Provisional application Ser. No. 63/488,412, titled “Dynamic Shunt Systems,” filed by Samuel Robert Browd, et al., on Mar. 3, 2023.
U.S. application Ser. No. 18/313,249, titled “Dynamically Controlled Cerebrospinal Fluid Shunt,” filed by Samuel Robert Browd, et al., on May 5, 2023
U.S. Application Ser. No. 63/365,407, titled “Distributed Sensing and Control of Cerebrospinal Fluid,” filed by Samuel Robert Browd, et al., on May 26, 2022
U.S. Application Ser. No. 63/477,158, titled “Central Nervous System Monitoring and Intervention,” filed by Samuel Robert Browd, et al., on Dec. 23, 2022
U.S. Application Ser. No. 63/590,313, titled “Neurosurgical Devices and Methods,” and filed by Samuel Robert Browd, et al., on Oct. 13, 2023
U.S. Application Ser. No. 63/590,191, titled “Dynamic Guided Physician-Patient Interaction Engine,” and filed by Samuel Robert Browd, et al., on Oct. 13, 2023

This application incorporates the entire contents of the foregoing application(s) herein by reference.

### TECHNICAL FIELD

Various embodiments relate generally to methods and apparatus for an implantable physiological sensor, particularly for monitoring cerebrospinal fluid (CSF) pressure and electroencephalogram (EEG) signals within the brain.

### SUMMARY

Apparatus and associated methods relate to a multimodal signal management system (MMSMS) that includes two or more electrodes, and a cerebrospinal fluid pressure (CSF) monitor enclosed within a housing implanted in the brain. In an illustrative example, the MMSMS, may, for example, include a conduit configured to provide fluid communication between the CSF-containing region in the brain and a remote location. Various embodiments may advantageously provide optional therapeutical CSF draining while monitoring electroencephalogram (EEG) and CSF pressure waveform information.

In various embodiments, a multimodal signal management system may include at least two electrodes and a cerebrospinal fluid (CSF) pressure monitor enclosed within a housing that is implanted in the brain. The system enables simultaneous EEG monitoring, CSF pressure measurement, and therapeutic CSF drainage through a conduit that provides fluid communication between the CSF-containing region in the brain and a designated drainage location.

Various embodiments may advantageously regulate CSF drainage relative to EEG signals, which may advantageously enhance researchers and healthcare providers ability to explore relationships between CSF pressure dynamics and neurological activity. The MMSMS may also provide automated diagnostic support and therapeutic interventions, enhancing patient treatment in trauma and neurosurgical applications.

Various embodiments may achieve one or more advantages. For example, some embodiments may advantageously modulate CSF drainage relative to EEG signals. In some implementations the MMSMS may enable the ability to research connections between CSF pressure and EEG signals. In some embodiments, the MMSMS may provide data that health care providers may use to diagnose or treat the patient.

The details of various embodiments are set forth in the accompanying drawings and the description below. Other features and advantages will be apparent from the description and drawings, and from the claims.

### BRIEF DESCRIPTION OF THE DRAWINGS

FIG. 1A depicts an exemplary multimodal signal management system (MMSMS) architecture employed in an illustrative use-case scenario.

FIG. 1B depicts a close-up schematic of the illustrative use-case scenario in FIG. 1A surgically implanted in a patient. FIG. 2A depicts a schematic of an exemplary embodiment of an MMSMS.

FIG. 2B depicts a schematic of another exemplary embodiment of an MMSMS.

FIG. 3 depicts a schematic of another exemplary embodiment of an MMSMS.

FIGS. 4A-4C depict partial views of exemplary embodiments of a catheter capable of use with various embodiments of the MMSMS.

FIG. 5 is a block diagram depicting an exemplary MMSMS in operative contact with a dura of a patient's brain.

FIG. 6 is a flowchart illustrating exemplary operations that may be performed by a multimodal signal management engine (MMSME) in the MMSMS of FIG. 5.

Like reference symbols in the various drawings indicate like elements.

### DETAILED DESCRIPTION OF ILLUSTRATIVE EMBODIMENTS

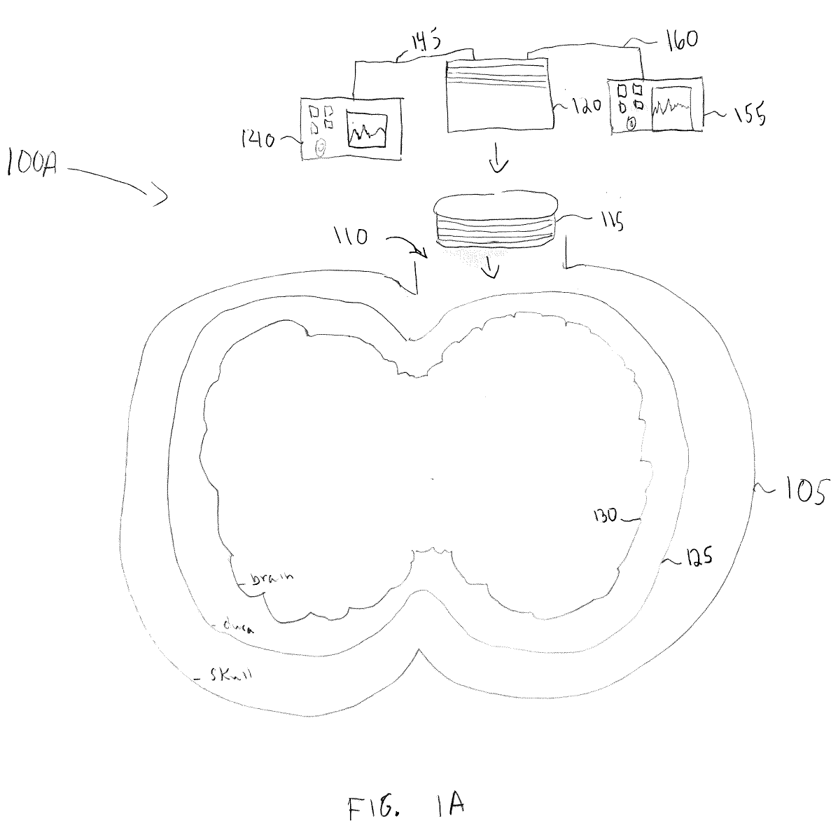

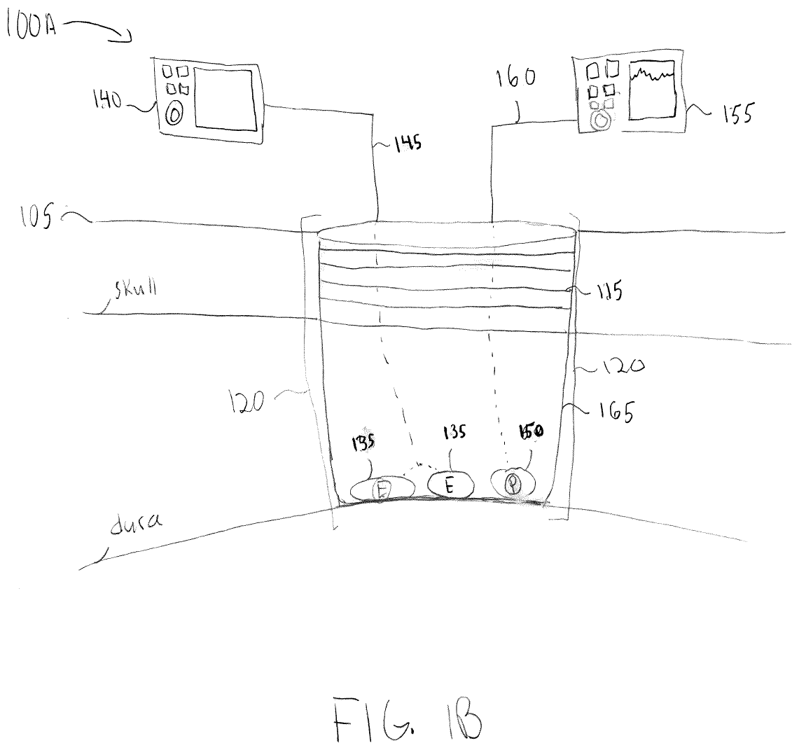

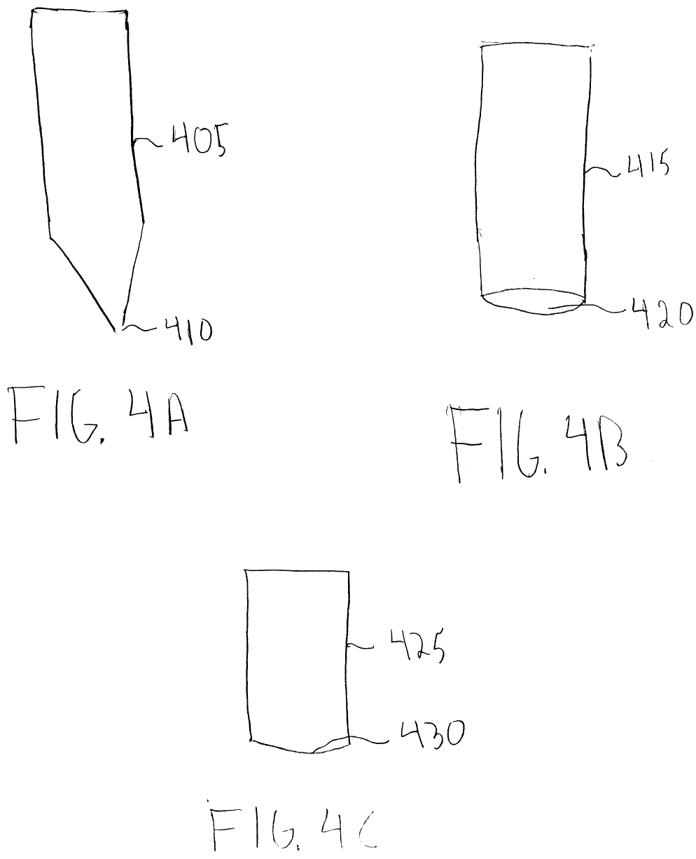

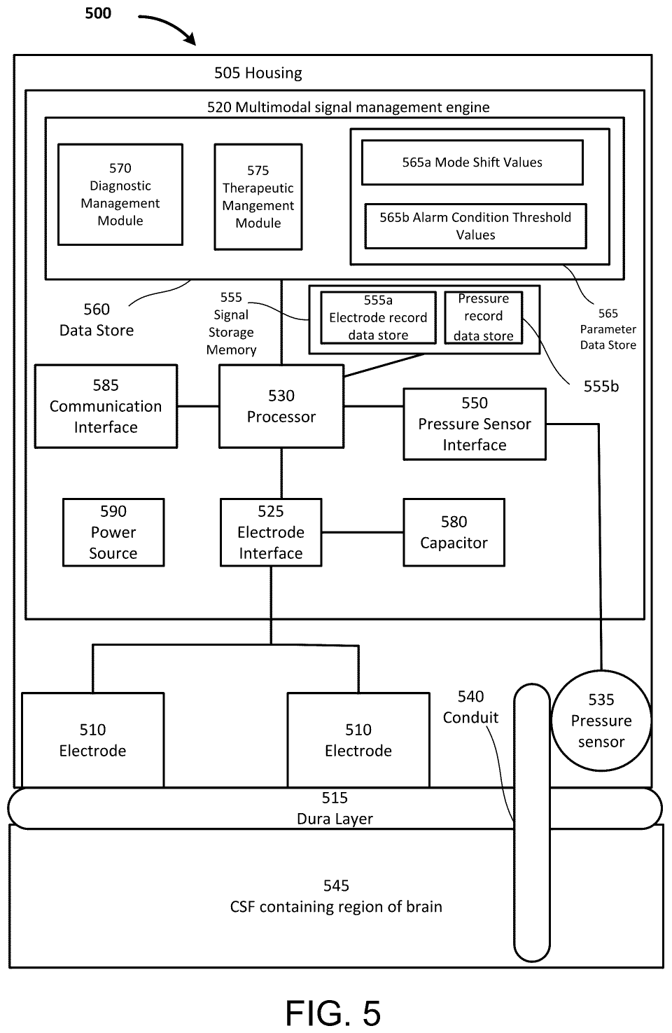

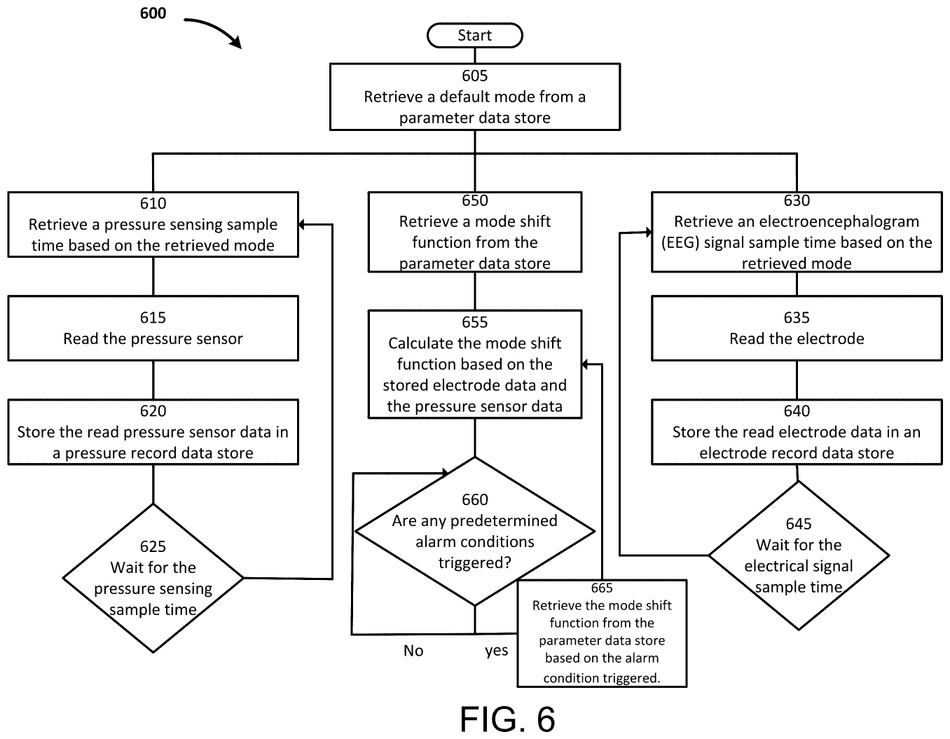

To aid understanding, this document is organized as follows. First, to help introduce discussion of various embodiments, a multimodal signal management system (MMSMS) is introduced with reference to FIGS. 1-3. Second, exemplary embodiments of a catheter are described with reference to FIGS. 4A-4C. Third, with reference to FIG. 5, a block diagram of an exemplary MMSMS deployed in vivo is presented. Lastly, with reference to FIG. 6, the discussion turns to a flowchart of an exemplary method of operations that may be performed by a multimodal signal management engine (MMSME), such as the embodiment introduced with reference to FIG. 5. Finally, the document discusses further embodiments, exemplary applications and aspects relating to a MMSMS.

FIG. 1A depicts an exemplary multimodal signal management system (MMSMS) architecture employed in an illustrative use-case scenario. As shown in FIG. 1A, an illustrative surgical scenario 100A may occur, for example, when a surgeon acts to provide a patient skull 105 with an implantable sensor system, for example, based on trauma to the patient. In the exemplary scenario 100A, the surgeon may create in the patient's skull 105 an aperture 110. The aperture 110 is configured to receive a ferrule 115. In this example, the depicted ferrule 115 has outer threads for engaging a burr hole in the skull 105, and inner threads for positioning a MMSMS 120. The MMSMS 120 includes outer threads for engaging the corresponding inner threads on the ferrule 115. In various implementations, the inner threads of the ferrule 115 may be configured to precisely control the linear position of the MMSMS 120, for example, which may advantageously permit the surgeon to accurately control the amount of engagement (e.g., contact pressure and/or interference) between a proximal end of the MMSMS 120 and a dura 125 that substantially surrounds a brain 130 in the skull 105.

This illustrative use-case scenario 100A may represent, for example, a trauma surgical environment in which a surgeon (not shown) may be preparing to install the MMSMS 120 via threaded engagement with the ferrule 115. As will be described in further detail with reference, for example, to at least FIGS. 5-6, the MMSMS 120 includes pressure sensing and electroencephalogram (EEG) monitoring capabilities. In various implementations, the installed MMSMS 120 may record both EEG waveform information and cerebrospinal fluid (CSF) pulsatile pressure waveform information and may store the captured waveform information over a predetermined period of time.

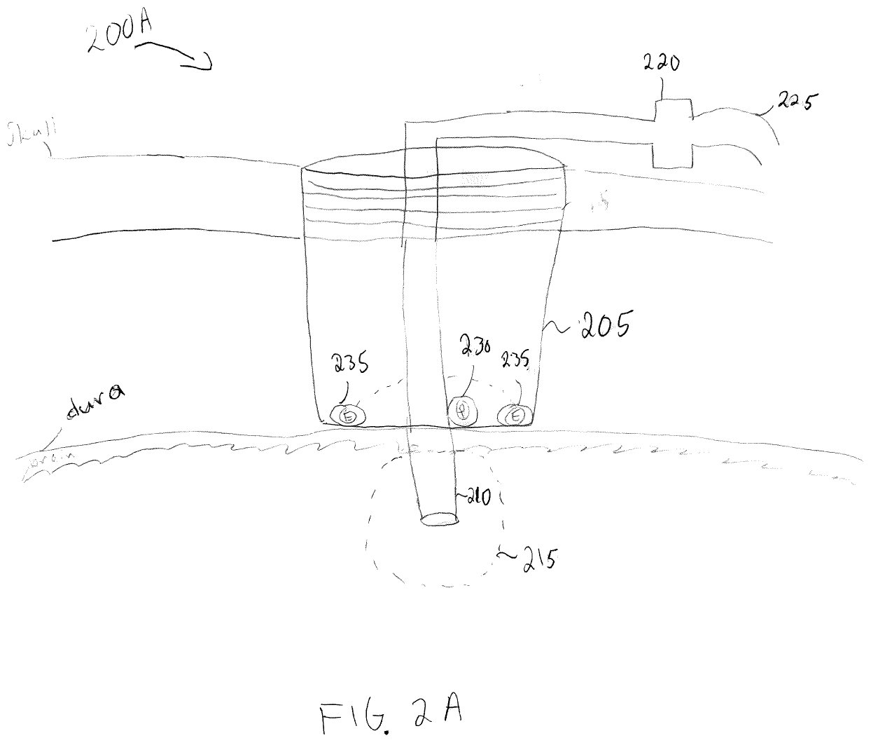

In some embodiments, the MMSMS 120 may simultaneously provide EEG/CSF pressure monitoring while also providing fluid communication between the CSF-containing region in the brain 130 and a remote location. In some examples, the remote location may be external to the patient, such as in a trauma scenario (e.g., scenario 100A of FIG. 1A). In some implementations, the remote location may be another drainage location in the patient's body, such as in an ambulatory application (examples of which are described with reference to FIGS. 2A 2B and 3).

Various advantages may be achieved from the MMSMS 120 providing EEG and CSF pressure monitoring at the same time as providing fluid communication for aspirating CSF fluid, for example. In some embodiments, the MMSMS 120 may provide data that health care providers may use to diagnose or treat the patient. For example, brain health may be indicated by the monitored CSF pulsatile waveform information in association with EEG activity. Various embodiments may further provide continuous passive and/or regulated drainage of excess CSF, which may advantageously protect the patient's brain 130. In some embodiments, the MMSMS 120 may be configured to be fully implanted with internal drainage at a remote location, wherein the CSF fluid flow passes through the MMSMS 120. In some embodiments, pressure may be regulated by a valve or other flow restriction device. In some implementations the MMSMS 120 may provide control of one or more pressure regulating actuators, where the MMSMS 120 generates control signals as a function of monitored CSF pressure waveform data and/or EEG waveform information.

FIG. 1B depicts a close-up schematic of the illustrative use-case scenario in FIG. 1A surgically implanted in a patient. As depicted, the MMSMS 120 is implanted in the skull 105 via threaded engagement with the ferrule 115. The MMSMS 120 includes two or more electrodes 135 positioned at the proximal end of the MMSMS 120. The two or more electrodes 135 are in electrical contact with the dura 125 such that the two or more electrodes 135 may detect EEG activity generated within the brain 130. The two or more electrodes 135 transmit the detected EEG activity to an EEG monitoring device 140 via leads 145. The EEG monitoring device 140 may, for example, amplify, filter, digitize, store, perform signal processing and/or display the detected EEG activity into a digitized waveform. The two or more electrodes 135 may, for example, wirelessly transmit the detected EEG activity to an EEG monitoring device 140.

In this illustrative surgical scenario 100A, the MMSMS 120 includes at least one pressure sensor 150. The at least one pressure sensor 150 is positioned at a proximal end of the MMSMS 120. The at least one pressure sensor 150 is in mechanical communication with the dura such that it may detect pressure exerted on its sensing surface due to intracranial pressure variations. The intracranial pressure variations may, for example, include changes in CSF pressure. The intracranial pressure variations may, for example, include changes in brain swelling. As depicted, the at least one pressure sensor 150 transmits the detected changes in intracranial pressure to a pressure sensor monitor 155 via leads 160. The pressure sensor monitor 155 may, for example, amplify, filter, digitize, store, perform signal processing and/or display the detected changes in intracranial pressure into a digitized waveform. The at least one pressure sensor 150 may, for example, wirelessly transmit the detected changes in intracranial pressure to a pressure sensor monitor 155.

In this illustrative surgical scenario 100A, the MMSMS 120 encloses the at least one pressure sensor 150 and the two or more electrodes 135 into a housing 165. The housing 165 may, for example, fit within a burr hole. By way of example, and not limitation, the housing 165 may have a diameter of about 1.5 millimeters. The housing 165 enclosing the at least one pressure sensor 150 and the two or more electrodes 135 in one unit may advantageously enable the MMSMS to monitor the brain's EEG activity and intracranial pressure during a brain trauma event in a minimally invasive manner.

FIG. 2A depicts a schematic of an exemplary embodiment of an MMSMS 200A. In the depicted embodiment, the MMSMS 200A is a fully implanted implementation in which the MMSMS 200A and any attached drainage conduit are located subcutaneously such that the patient may, for example, be untethered to leads or tubing. This may be advantageous for ambulatory long-term applications.

The MMSMS 200A includes a housing 205. The housing 205 is implanted in a skull via threaded engagement with the skull. The housing 205 includes a conduit 210. The conduit 210 may, for example, provide a catheter. The conduit 210 may, for example, provide an external ventricular drain (EVD). The conduit 210 is disposed in a CSF-containing region of the brain 215. The conduit 210 is configured to be in fluid communication with the CSF-containing region of the brain 215. The conduit 210 is operatively coupled to a valve 220. The conduit 210 extends radially from the housing 205 to the valve 220. The conduit may, for example, extend subcutaneously. The valve 220 is configured to control the rate of CSF flow into the conduit 210. The valve operatively couples tubing 225 to the conduit 210. CSF may, for example, flow up through the conduit 210 and the tubing 225 to a remote location. The remote location may, for example, include another drainage location in the patient's body. The remote location may, for example, include a location external to the patient.

The conduit 210 is operably coupled to an at least one pressure sensor 230. The valve 220 may, for example, halt the drainage of CSF out of the conduit such that the pulsatile CSF pressure within the conduit 210 may represent the pulsatile CSF pressure within the CSF-containing region of the brain 215. The at least one pressure sensor 230 may, for example, measure the pulsatile CSF pressure within the brain by measuring the pulsatile CSF pressure in the conduit 210. This exemplary MMSMS 200A may advantageously enable the MMSMS to measure intracranial pressure while therapeutically draining CSF.

FIG. 2B depicts a schematic of another exemplary embodiment of an MMSMS 200B. The MMSMS 200B includes an at least one pressure sensor 230 operatively coupled to a proximal end of the housing 205. The at least one pressure sensor 230 is positioned within the CSF-containing region 215. The at least one pressure sensor 230 positioned within the CSF-containing region 215 may, for example, advantageously enable continuous measurement of pressure within the CSF-containing region 215 of the brain without halting the drainage of CSF out of the conduit 210, which may enable its use in an ambulatory setting.

In the exemplary MMSMS 200A and 200B, the two or more electrodes 235 are configured in a substantially similar arrangement to the two or more electrodes 135 as described above with reference to FIGS. 1A and 1B.

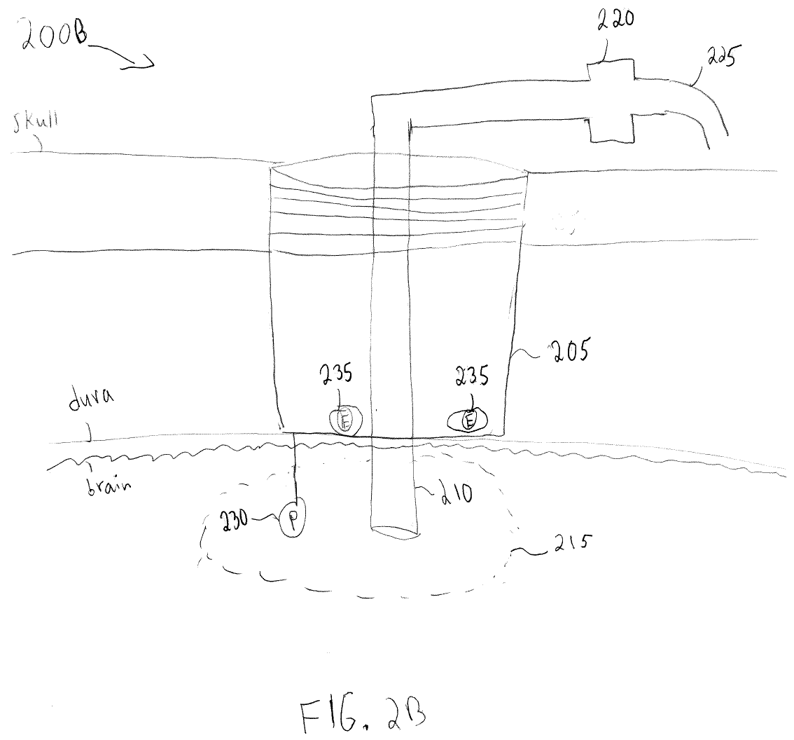

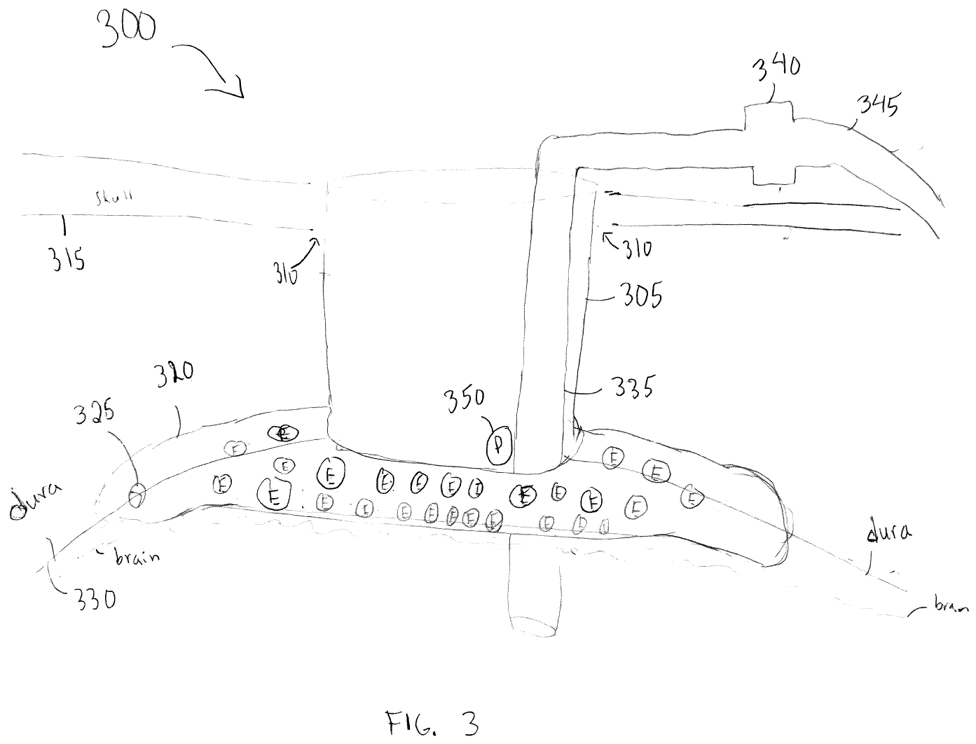

FIG. 3 depicts a schematic of another exemplary embodiment of an MMSMS 300. The MMSMS 300 includes a housing 305. The housing 305 is implanted within an aperture 310 in a skull 315. The housing includes an annular flex circuit 320. The annular flex circuit 320 operatively couples an array of electrodes 325. The array of electrodes 325 are in electrical contact with a dura 330. The annular flex circuit 320 may, for example, be flexible. The flexibility of the annular flex circuit 320 may permit the annular flex circuit 320 to conform substantially to a nonplanar surface region of the dura 330. The annular flex circuit 320 may advantageously enable the array of electrodes 325 to cover a large surface area of the dura 330. The annular flex circuit 320 may advantageously enable the array of electrodes 325 to multidimensionally monitor EEG signals from the brain. The array of electrodes 325 may, for example, include individual electrodes angled in different directions to monitor EEG signals according to a preferred direction such as by beam steering techniques. This MMSMS 300 may, for example, be advantageously employed within an ambulatory setting.

In this exemplary MMSMS 300, the conduit 335, valve 340, tubing 345, and pressure sensor 350 may be substantially similar to the respective conduit 210, valve 220, tubing 225, and pressure sensor 230 as described above with reference to FIGS. 2A and 2B.

In some implementations of the MMSMS 300, a system may include multiple MMSMSs 300 implanted in the brain. The multiple MMSMSs 300 may, for example, be communicably coupled via wired or wireless techniques.

FIGS. 4A-4C depict partial views of exemplary embodiments of a catheter capable of use with various embodiments of the MMSMS. FIG. 4A includes a catheter 405 with a pointed open tip 410. The pointed open tip 410 may advantageously enable the catheter to puncture the dura and to be positioned within a CSF-containing region of the brain. FIG. B includes a catheter 415 with a rounded open tip 420. The rounded open tip 420 may advantageously enable the catheter 415 to fill with CSF quickly. FIG. 4C includes a catheter 425 with a flexible membrane pressure sensing tip 430. The flexible membrane pressure sensing tip 430 may advantageously enable pressure measurement, for example, without an additional pressure sensor.

FIG. 5 is a block diagram depicting an exemplary MMSMS 500 in operative contact with a dura of a patient's brain. The MMSMS includes a housing 505. The housing 505 encloses two or more electrodes 510. The two or more electrodes 510 are in electrical contact with a dura layer 515. The two or more electrodes 510 measure the EEG activity of the brain while in electrical contact with the dura layer 515. The two or more electrodes 510 transmit a signal representing the EEG activity of the brain to a multimodal signal management engine (MMSME) 520 via an electrode interface 525 within the MMSME 520. The electrode interface 525 communicably couples the two or more electrodes 510 such that the two or more electrodes 510 transmit the signal to the electrode interface 525. The electrode interface 525 transmits the signal to the processor 530 of the MMSME 520.

The housing encloses at least one pressure sensor 535. The at least one pressure sensor 535 operably couples a conduit 540. The conduit 540 is positioned in a CSF containing region of the brain 545 via an aperture in the dura layer 515. The conduit 540 draws in CSF from the CSF containing region of the brain 545. The conduit 540 may, for example, provide fluid communication between the CSF-containing field in the brain and a remote location. The remote location may include, for example, a location external to the patient's body for CSF to drain. The remote location may include, for example, an internal body system within the patient for CSF to drain. The at least one pressure sensor 535 measures the pressure within the conduit 540. The pressure measurement within the conduit 540 represents the CSF pressure within the brain. The at least one pressure sensor 535 transmits a signal representing the CSF pressure within the brain to the MMSME 520 via a pressure sensor interface 550 within the MMSME 520. The pressure sensor interface 550 communicably couples at least one pressure sensor 535 such that at least one pressure sensor 535 transmits the signal to the pressure sensor interface 550. The pressure sensor interface 550 transmits the signal to a processor 530.

The processor 530 is coupled to a signal storage memory 555. The processor 530 may, for example, transmit the signal representing the EEG activity of the brain and the signal representing the CSF pressure within the brain to a signal storage memory 555. The signal storage memory 555 includes an electrode record data store 555a. The processor 530 may transmit the signal representing the EEG activity of the brain 545 to the electrode record data store 555a. The electrode record data store 555a may accumulate a time series of data from the EEG activity measurements such that the processor can store individual values of measurements of EEG activity within the electrode record data store 555a. The signal storage memory includes a pressure record data store 555b. The processor may transmit the signal representing the CSF pressure within the CSF containing region of the brain to the pressure record data store 555b. The pressure record data store 555b may accumulate a time series of data from the CSF pressure measurements such that the processor can store individual values of measurements of CSF pressure within the pressure record data store 555b.

The processor 530 is coupled to a data store 560. The processor 530 may, for example, transmit the signal representing the EEG activity of the brain and the signal representing the CSF pressure within the brain to the data store 560. The data store 560 includes a parameter data store 565. The parameter data store 565 includes mode shift values 565a. The mode shift values 565a may, for example, include signal collection times that determine how often the two or more electrodes 520 and the at least one pressure sensor 535 measure a signal. The mode shift values 565a may, for example, include a default mode. The default mode may, for example, include a signal collection time that is present when the MMSMS 500 is not in a mode function based on an alarm condition. The parameter data store 565 includes one or more alarm condition threshold values 565b. The alarm condition threshold values 565b may include pressure threshold values and EEG activity threshold values that when reached, cause the processor 530 to generate an alarm condition. By way of example, and not limitation, an exemplary pressure threshold value may, for example, include a CSF pressure in the brain above 20 millimeters of mercury. By way of example, and not limitation, an exemplary EEG activity threshold value may, for example, include delta power of 100 μV²/Hz in frontal regions during wakefulness. By way of example, and not limitation, an exemplary EEG activity threshold value may, for example, include spike-wave discharges at 3 Hz in the frontal lobe. The parameter data store 565 may, for example, transmit a signal to the processor 530 to alert the processor an alarm condition is triggered. In some embodiments, the processor 530 may retrieve a mode shift function from the parameter data store 565 based on the triggered alarm condition.

The processor 530 may, for example, retrieve a mode shift function from the parameter data store 565. The parameter data store may, for example, include electrode sample times. The parameter data store may, for example, include pressure sensing sample times. The processor 530 may, for example, retrieve a mode shift function from a diagnostic management module 570 within the data store 560. The diagnostic management module 570 may, for example, include diagnostic information about a patient based on the recorded signal representing the EEG activity of the brain and the recorded signal representing the CSF pressure of the brain. The processor 530 may, for example, retrieve a mode shift function from a therapeutic management module 575 within the data store 560. The therapeutic management module 575 may, for example, include instructions about treating a patient based on the recorded signal representing the EEG activity of the brain and the recorded signal representing the CSF pressure of the brain. For example, the therapeutic management module 575 may include instructions, that when executed by the processor 530, may perform operations to cause a capacitor 580 to discharge through the two or more electrodes 510 an electrical charge for a predetermined therapeutic purpose.

The processor 530 may, for example, transmit a signal to a communication interface 585. The communication interface 585 may, for example, be in operative communication (e.g., over a wired, wireless, or optical communication link) to an external data processing device that may be configured to log data received from the MMSMS. The external data processing device may, for example, include a display device to display the electrode data and the pressure data, for example, relative to a common time axis. The external device may, for example, include an external medical device configured to provide treatment to a patient based on the readings of the MMSMS 500.

FIG. 6 is a flowchart illustrating exemplary operations that may be performed by a multimodal signal management engine (MMSME) in the MMSMS of FIG. 5. In the depicted Figure, an exemplary set of operations 600 for monitoring the MMSMS multi-modal sensors is disclosed. By way of example and not limitation, and with reference to FIG. 5, the set of operations may be included in a program of instructions stored in, for example, the diagnostic management module 570. In operation, the MMSMSE 520 may perform operations to monitor EEG and CSF waveform signals in response to the processor 530 executing the instructions stored in the diagnostic management module 570.

The set of operations 600 begins at step 605, in which a processor of an MMSME retrieves a default mode from a parameter data store. The default mode may, for example, include a predetermined signal collection time that is present when a MMSMS is not in a mode function based on an alarm condition.

At step 610, the processor retrieves a pressure sensing sample time based on the retrieved mode in step 605. At step 615, the processor reads a pressure sensor and retrieves pressure sensor data. At step 620, the processor sends the read pressure sensor data to a pressure record data store for the pressure sensor data to be stored. At step 625, the processor waits for the pressure sensing sample time, and then reverts to step 610.

Concurrently, at step 630, the processor 530 retrieves an EEG signal sample time based on the retrieved mode from step 105. At step 635, the processor reads an electrode and retrieves electrode data. At step 640, the processor sends the read electrode data to an electrode data store for the electrode data to be stored. At step 645, the processor waits for the EEG signal sample time, and then reverts to step 630.

Concurrently, at step 650, the processor 530 retrieves a mode shift function from the parameter data store. At step 655, the processor 530 calculates the mode shift function based on the stored electrode data and the stored pressure sensor data. At step 660, the processor determines if any predetermined alarm conditions have been triggered based on the electrode data and the pressure sensor data. If no, the processor remains at step 660. If yes, then at step 665, the processor retrieves the mode shift function from the parameter data store based on the alarm condition triggered.

Although various embodiments have been described with reference to the figures, other embodiments are possible.

In some implementations, the MMSMS of, for example, the embodiments described with reference to FIG. 1B or 2B, may further be adapted to drain the CSF fluid to an external location while monitoring the CSF pressure and EEG activity.

In some implementations, the MMSMS may, for example, externally transmit recorded EEG and CSF pressure data in its raw form, providing direct electrode readings and pressure sensor measurements for real-time monitoring or further analysis. The MMSMS may process the recorded EEG and CSF pressure data via a processor that applies a predetermined algorithm stored in data store. The processed data may, for example, be transmitted externally to indicate specific health conditions, such as an alarm condition triggered by abnormal EEG patterns or CSF pressure levels. The MMSMS may, for example, externally transmit the processed data to assess the overall health status of a patient. Additionally, the MMSMS may be configured to minimize power consumption to extend the useful charge of the battery.

In some implementations the MMSMS may, for example, be configured for minimally invasive implantation. The MMSMS may be securely housed within a ferrule-threaded enclosure such that the MMSMS interfaces a patient's skull. The MMSMS may, for example, provide real-time multimodal data acquisition through electrodes for EEG monitoring, CSF pressure sensors to detect intracranial pressure variations, and a drainage conduit for passive regulated CSF removal. In some embodiments, the system's processor may, for example, synchronize EEG and CSF pressure data, enabling correlation between brain activity and fluid dynamics. In some implementations, machine-learning algorithms may, for example, analyze data trends to predict abnormal conditions, triggering preemptive alerts or therapeutic responses.

Various embodiments may provide various advantages. For example, the MMSMS may, uniquely integrate EEG and CSF pressure sensing within a single implantable unit. Some embodiments may provide for concurrent draining of excess CSF, alone or combination with injection with prescribed materials (e.g., pharmaceuticals, nutrients, diagnostic contrast materials, etc.) Some embodiments may include therapeutic capabilities such as actuated control of CSF drainage based on detected EEG anomalies or pressure thresholds. In some implementations, the MMSMS advantageously enables real-time patient monitoring through a remote interface. The MMSMS may include configurations such as annular flex circuits, for example, that enable broad-area EEG signal collection, enhancing diagnostic accuracy. In some embodiments, the processor may, for example, dynamically adjust sampling frequency in response to predefined alarm conditions (e.g., pressure spikes, abnormal EEG patterns).

In some implementations, the MMSMS may be employed in an exemplary method for monitoring and regulating intracranial pressure and EEG activity. The method may, for example include: collecting real-time CSF pressure waveforms via at least one implantable pressure sensor; detecting EEG activity via at least two dura-contact electrodes; storing and analyzing sensor data to detect abnormalities; automatically adjusting CSF drainage rate in response to detected conditions; and/or transmitting EEG and CSF pressure data wirelessly to a remote medical interface.

In some embodiments the MMSMS may, for example, include a processor-controlled CSF drainage system that dynamically modulates fluid flow in response to EEG signal fluctuations. In some implementations, the MMSMS includes, for example, an implantable multimodal sensor with configurable electrode orientations to enhance EEG signal detection from multiple brain regions. In some implementations, the MMSMS includes, for example, a wireless communication module configured to interface with external medical systems, enabling real-time data access.

Although exemplary methods and apparatus have been described with reference to FIGS. 1-6, other implementations may be deployed in other industrial, scientific, medical, commercial, and/or residential applications. For example, the MMSMS may be deployed in animal studies to research a connection between CSF pressure within the brain and EEG activity. For example, the MMSMS may be deployed to modulate sleep. In some embodiments, the EEG activity can be compared to CSF pulsatility via pressure waves to manage sleep and CSF movement.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single-use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces, and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A system comprising:
a housing, the housing configured to operably contact a dura while implanted through an aperture in a patient's skull, the housing providing an enclosure for a multimodal signal management engine (MMSME), the MMSME comprising:
at least one pressure sensor configurable to monitor a cerebrospinal fluid (CSF) pulsatile pressure in a CSF-filled region of a brain;
two or more electrodes configured to be in electrical contact with the dura and monitor an electroencephalogram (EEG) signal in the patient's brain;
a processor operatively coupled to the at least one pressure sensor and the two or more electrodes; and
a datastore containing instructions that, when operated by the processor, cause operations to be performed to collect a CSF pressure signal waveform and an EEG signal waveform over a predetermined period.

2. The system of claim 1, wherein the operations further comprise retrieving pressure sensor data representing the patient's CSF waveform signal.

3. The system of claim 1, wherein the operations further comprise retrieving electrode data representing the patient's EEG waveform signal.

4. The system of claim 1, wherein the operations further comprise retrieving a mode shift function from a parameter data store.

5. The system of claim 4, wherein the mode shift function is calculated based on stored pressure sensor data and stored electrode data.

6. The system of claim 4, wherein the mode shift function is retrieved based on a triggered predetermined alarm condition.

7. The system of claim 1, further comprising a ferrule having outer threads configured for fixedly engaging the ferrule in a burr hole of a skull, and inner threads configured to threadedly engage the housing, wherein the housing is axially positionable with respect to the dura by selective rotation of the housing while engaged in the ferrule.

8. The system of claim 1, wherein the two or more electrodes are communicably coupled to an electrode interface, wherein the electrode interface is positioned within the MMSME and electrically coupled to a capacitor, such that the electrode interface is configured to provide an electrical charge to the two or more electrodes via the capacitor.

9. The system of claim 1, wherein the housing further comprises an annular flex circuit, wherein the two or more two electrodes are operatively coupled to the annular flex circuit.

10. The system of claim 1, wherein the housing further comprises a catheter positioned at the base of the housing, wherein the catheter is configured to provide fluid communication from a CSF-filled region in the brain into the housing via an aperture in the dura.

11. The system of claim 10, wherein the catheter extends the fluid communication path distally to an external reservoir for drainage.

12. The system of claim 10, wherein the catheter provides a fluid communication path that extends through the housing and radially from the housing into a subcutaneous path to a location within the body for drainage.

13. The system of claim 10, further comprising a valve, the valve positioned radially outside from the MMSME housing, such that the valve is operably coupled to the catheter, wherein the valve is configured to control the rate of CSF drainage from the catheter.

14. The system of claim 10, wherein the at least one pressure sensor is positioned in the housing such that the at least one pressure sensor is operably coupled to the catheter, wherein the at least one pressure sensor is configured to monitor the pulsatile pressure of CSF within the catheter.

15. The system of claim 1, wherein the at least one pressure sensor is positioned in a subdural space such that the at least one pressure sensor is configured to provide fluid communication from a CSF-filled region in the brain into the housing via an aperture in the dura.
